(19) United States
(12) Patent Application Publication
(10) Pub. No.: US 2014/0045885 A1
(43) Pub. Date: Feb. 13, 2014

(54) Decoquinate, 4-hydroxyquinolones and napthoquinones combined with levamisole, Imidazothiazole, for the prevention and treatment of sarcocystosis and equine protozoal myeloencephalitis caused by Sarcocystis and Neospora and other apicomplexan protozoans.

(71) Applicant: Siobhan P. Ellison, (US)

(72) Inventor: Siobhan P. Ellison, (US)

(21) Appl. No.: 13/571,558

(22) Filed: Aug. 10, 2012

(51) Int. Cl. A61K 31/47; A61K 31/429; A61P 25/00; A61K 31/122; A61P 33/02; A61P 15/06

(52) U.S. Cl. 514/312; 514/682; 514/368

(57) ABSTRACT

Apicomplexan parasites that infect horses such as Sarcocystis sp., Sarcocystis neurona and Neospora hughesi may be killed with decoquinate, a 4-hydroxyquinolone and/or a naphthoquinone and enhanced effects for treatment of animal disease are seen with the addition of levamisole, imidazothiazole. Based on such parasite-killing activity, these compounds are used in methods of preventing and treating infections, neurological disease or dysfunction such as protozoal myeloencephalitis, especially equine protozoal myeloencephalitis.

### FIELD OF THE INVENTION

The invention generally relates to infection or neurologic disease and dysfunction, and particularly relates to apicomplexan parasites causing equine neurologic syndromes.

### BACKGROUND OF THE INVENTION

Equine protozoal myeloencephalitis (EPM) is a neurologic syndrome in horses from the Americas and is usually caused by infection with the apicomplexan parasites, Sarcocystis neurona and Neospora hughesi but can be caused by Sarcocystis fayeri and Sarcocystis falcatula. Infections with virulent Sarcocystis sp. precede EPM and EPM is a devastating sequel to infections. EPM is considered the most important protozoal disease of horses in the United States, and usually is considered in any horse with neurological signs. Serological surveys using Peptide ELISA tests demonstrate that about 48% or more of horses have antibodies to S. neurona indicating high exposure to that parasite. There are about 7.2 million horses in the United States, corresponding to a $120 billion dollar annual industry. Clinical EPM occurs in 0.88% of horses.

In a recent survey representatives of the horse industry, including veterinarians and horse owners, of the infectious diseases listed, EPM was listed by 24% and ranked first (USDA, APHIS Report May, 1997, and 2001). The number of cases of EPM diagnosed in horses with neurological signs at the Ohio State University veterinary school increased from 24.9% in 1992 to 50% in 1996 indicating an increasing prevalence. Despite the release of toltrazuril (Marquis) for the treatment of EPM in horses the morbidity of EPM in the equine population has not changed in 17 years (Morbidity and Mortality report Frank Andrews EPM special session 2011).

The Virginia opossum, Didelpllis virginiana is the only known definitive host in North America. Dubey, J. P., Lindsay, D. S., 1998, “Isolation of Sarcocystis neurona from opossum (Didelphis virginiana) faeces in immunodeficient mice and its differentiation from Sarcocystisfalcatula, Int. J. Parasitol. 29,1823-1828. The nine-banded armadillo, Dasypus novemcinctus, is a natural intermediate host (Cheadle et al., 2001) and domestic cats (Felis domesticus) are experimental intermediate hosts (Dubey et al., 2000). Horses become infected by ingesting S. neurona sporocysts excreted in opossum feces. Opossums are also the definitive host for S. falcatula while dogs are the definitive hosts for S. fayeri.

Horses become infected with EPM-causing agents by ingesting sporocysts or oocysts while grazing or from contaminated feed or water. It is virtually impossible to prevent horses from encountering EPM-causing agents.

Conventionally, pyrimethamine and sulfonamides are used to treat EPM, with a prolonged course of treatment (twelve weeks being about the average length of treatment time). Usual treatment involves the use of sulfadiazine at a dose of 20 mg/kg, once or twice a day. In addition, affected horses are placed on pyrimethamine, at a dosage of 1.0 mg/kg daily for 120 days or longer. Duration of treatment may be longer if the CSF remains positive and/or the horse continues to demonstrate clinical signs of neurological disease. Complications of anemia and/or leukopenia have been observed, especially for doubling the pyrimethamine dose, and in some horses diarrhea occurs.

For these conventional therapies, a determination to discontinue treatment is based on either significant improvement of the clinical signs or the horse returning to normal and Western blot testing of CSF returning to negative. The combination of sulfadiazine and pyrimethamine results in a sequential blockade of folic acid metabolism. The efficacy of sulfadiazine and pyrimethamine is 45% in horses and the relapse rate is 60%. Strains of Sarcocystis neurona have been identified that are resistant to this anti-protozoal drug.

The specific concentration of pyrimethamine required to achieve an anti-protozoal level for S. neurona is not known.

Diclazuril (Clinicox, Pharmacia Upjohn, Canada, Protazil, Intervet-Schering Plough, United States), a coccidiostat, is an alternative treatment for horses not responding to the above-mentioned traditional therapies or in horses having developed complications. The drug is absorbed quickly and has been found in serum one hour after feeding to horses. It is a triazine and has been used as a prophylactic agent against coccidiosis in poultry and has been used experimentally in the treatment of similar problems in rabbits. It has anti-S. neurona activity in cell cultures infected with S. neurona. The efficacy of diclazuril is 60% in horses to improve one grade of disease with a relapse rate of 60% and a cure rate of less than 25%. Strains of Sarcocystis neurona have been identified that are resistant to this anti-protozoal drug.

Toltrazuril (Baycox 5% suspension; Bayer, Canada, Marquis, Bayer, United States) is an anti-coccidial drug used in several species. The mechanism of action is to disrupt intracellular pathways important in energy metabolism as well as cell division. This drug and its major metabolite, ponazuril have potential efficacy for the treatment of EPM. Toltrazuril appears to have good oral absorption and fairly long elimination time (48-72 hours).

The drug has good lipid solubility and is well absorbed into CSF. In horses given toltrazuril at 5 mg/kg daily for 10 days, plasma levels of toltrazuril were 20 mcg/ml with a mean CSF concentration of 160 mcg/ml. The use of this drug has not been shown to result in any complications nor have elevations of serum chemistry values or changes in complete blood counts been observed. After FDA approval and 10 years of commercialization the efficacy of toltrazuril is 60% in horses to improve one grade of disease with a relapse rate of 60% and a cure rate of less than 25%. Strains of Sarcocystis neurona have been identified that are resistant to this anti-protozoal drug.

For horses treated with the above-mentioned conventional therapies, relapse may occur. For example, relapse may occur if horses are not treated long enough. Reactivation of the infection may occur during periods of unusual stress. The conventional chemotherapy regimens do not completely remove all disease-causing parasites from the central nervous system or the viscera of the animal. Also, if too-low a concentration of the conventional drugs is used, intracellular stages of the parasites are not killed. The relapse problem has prompted use of the above-mentioned conventional therapies at 2, 5, and 7 times the approved label doses for extended periods of time with no shown improvement in efficacy.

However, even if in most horses that contract EPM relief ultimately can be provided, the costs of treating EPM are substantial. Diagnostic neurological evaluation may cost $456 per horse. Treatment of horses for EPM can be expensive, especially since most affected horses are treated for a period of 120 to 150 days, and sometimes longer.

The monthly cost of treatment is approximately $2000.00 for a 450 kg horse. Reevaluation of the horse at 30 to 60 day intervals and a subsequent spinal tap at 90 to 120 days after initiation of treatment adds to the cost. If clinical signs persist, therapy is continued and reevaluated every 30 days. Treating horses with toltrazuril is estimated at $2000 (5 mg/kg) to $7000 (10 mg/kg). In addition to direct treatment costs, indirect costs also are associated with EPM, such as decreased performance time, loss of stake payments, transport costs, death or euthanasia.

Thus, improved treatments for equine EPM are wanted, as are methods for avoiding EPM in the first instance. In treating EPM, for example, there remains the hope that horses can be restored to health more rapidly than with current treatments and with more permanent results.

### SUMMARY OF THE INVENTION

The present invention exploits the discovery that decoquinate and particularly, decoquinate in conjunction with levamisole is highly active against S. neurona, and the further discovery that decoquinate even at low concentrations accomplishes intracellular stage killing in the horse. According to the invention, decoquinate, a 4-hydroxyquinolone, and/or a naphthoquinone or pharmaceutically acceptable salts (e.g., anionic or cationic, hydrochlorides, ammonium, sodium, etc.), esters (Cl 12 alkyl) or other derivatives (e.g., amine) thereof may be fed prophylatically to horses, to prevent EPM, and may be administered to horses to treat EPM.

It is not surprising the decoquinate can kill protozoa in the intestine of horses, however it is novel and unexpected that decoquinate and or decoquinate and levamisole effects rapid clinical response and reduction in antibody indicating eradication of infections in horses. Current dogma indicates that decoquinate would have no effect on equine infections because the bio availability of the drug would not allow killing.

In order to accomplish these and other objects of the invention, the present invention in a preferred embodiment provides a method of killing Sarcocystis neurona and Neospora hughesi and Sarcocystis fayeri and Sarcocystis falcatula, comprising contacting a population comprising Sarcocystis neurona and/or Neospora hughesi and Sarcocystis fayeri and Sarcocystis falcatula with an agent selected from the group consisting of decoquinate, a 4-hydroxyquinolone and a naphthoquinone, and salts, esters and levamisole and salts or derivatives thereof. In one embodiment of the inventive killing method, the population may be in an equine host. In a further embodiment of the killing method, contacting includes the step of orally providing said equine host with said agent. In a further embodiment the killing method, contacting includes the step of orally providing said bovine host with said agent.

In another preferred embodiment, the invention provides a method of preventing or treating equine neurological disease or dysfunction, comprising administration to an equine of a pharmaceutically effective dose of an agent selected from the group consisting of decoquinate, a 4-hydroxyquinolone and a naphthoquinone, and salts, esters, and levamisole and salts or derivatives thereof. In a further embodiment of such an inventive method, the neurological disease or dysfunction may be associated with infection with an apicomplexan parasite. In another embodiment of the invention, the neurological disease or dysfunction may be protozoal myeloencephalitis. The apicomplexan parasite may be Sarcocystis neurona or Neospora hughesi or Sarcocystis fayeri or Sarcocystis neurona. In a further preferred embodiment, the invention provides a method used when the equine has not yet demonstrated symptoms associated with protozoal myeloencephalitis.

### DETAILED DESCRIPTION OF THE PREFERRED EMBODIMENTS OF THE INVENTION

In a first preferred embodiment, a parasite population comprising Sarcocystis neurona and/or Neospora hughesi is killed by being contacted with decoquinate, a hydroxyquinolone (such as 4-hydroxyquinolone) and/or a naphthoquinone or pharmaceutically acceptable salts (e.g., anionic or cationic, hydrochlorides, ammonium, sodium, etc.), esters (Cl 12 aLkyl) or other derivatives (e.g., amine) with or without levamisole. Such compounds are known, such as the compounds with structures set forth in FIGS. 1A-G. See also FIG. 47.7 and pages 966-967 of David S. Lindsay and Byron L. Blagburn, Antiprotozoan Drugs, Section 11 (“Chemotherapy of Parasitic Diseases”), Chapter 47.

Compounds for use in the present invention (hereinafter anti-EPM compounds or agents) include those shown in FIGS. 1A-G, but are not particularly limited thereto: Buquinolate (4-hydroxy-6,7-diisobutoxy-3-quinoline-carboxylic acid ethyl ester, C2oH27NO5), Decoquinate (6-declyoxy-7-ethoxy-4-hydroxy-3-quinoline-carboxylic acid ethyl ester, C24H3sNOs), Nequinate (7-(benzyloxy)-6-n-butyl-1, 4-dihydro-4-oxo-3-quinoline-carboxylic acid methyl ester, C22H23NO4), Buparvaquone (2-t7 [symbol] ans-(4-t-butylcyclo-hexyl) methyl-3-hydroxy-1, 4-naphthoquinone, C2IH2603), Parvaquone (2-cyclohexy-3-hydroxy-1, 4-naphthoquinone, CI6H1603) and Atovaquone (2-[trans-4-(4-chlorophenyl) cyclohexyl]-3-hydroxy-1, 4-naphthoquinone, CHjgClOg) and levamisole, levamisole in the L-isomer of D, L-tetramisole.

By way of non-limiting example, a commercially available example of decoquinate is Deccox which is an anticoccidial feed additive containing 6% decoquinate, marketed by Alpharma Inc. of Fort Lee, N.J. Alphapharma's FDA clearances are for Deccox R use for prevention of coccidiosis in cattle, goats, sheep and broiler chickens.

Alpharma's product sheet data indicate that Deccox R is non-toxic to horses. We have used decoquinate at 0.5 mG/kG and levamisole at 1 mG/kG alone and in combination in 150 horses with no adverse reactions to show safety at the desired dose for treatment.

The invention may be used for prevention and treatment of neurologic disease such as equine EPM. In the case of prevention, horses which may be exposed to agents that cause EPM are provided with a sufficient quantity of the anti-EPM agent of this invention to kill or immobilize the EPM disease causing agents. In the case of treatment of horses suspected to have EPM, administration of an anti-EPM agent according to the present invention should begin as quickly as possible after clinical signs of the disease are recognized. At least as good recovery is expected using the invention as for conventional treatments, which are said to result in successful recovery in less than 60% of the EPM-affected horses. In case, treatment or prevention, the anti-EPM agent may be administered orally as part of feed, or by other means such as injection. As demonstration we show experimental trial results below.

Regular (such as daily) feeding of the above-mentioned anti-EPM compounds to horses may have further beneficial effects, such as (1) prevention of abortions due to Neospora caninum, Neospora hughesi or Toxoplasma gondii; (2) prevention of equine babesiosis (because the piroplasmas have mitochondria that are sensitive to mitochondrial inhibitors); and (3) prevention of intestinal coccidiosis caused by Eimeria leuckarti in foals and other equids.

The following experimentation was conducted for Sarcocystis neurona isolates and cell culture by David Lindsay at the University of Virginia. These published experiments indicate the possible efficacy of decoquinate against S. neurona.

Sarcocystis neurona merozoites (SN2, SN3, or SN6 strains, isolated from a horse with EPM (Dubey et al., 2001) were grown and maintained in bovine turbinate (BT cells, ATTC CRL 1390, American Type Culture Collection) or African green monkey (Cercopithecus aethiops) kidney cells (CV-1 cells, ATTC CCL-70, American Type Culture Collection, Rockville, Md., USA) according to the method in Lindsay, D. S., and Dubey, J. P.,”Determination of the activity of diclazuril against Sarcocystis neurona and Sarcocystis falcatula in cell cultures, J. Parasitol. 86,164-166 (2000). The host cells were grown to confluence in 25 cm2 plastic cell culture flasks in growth media that consisted of 10% (v/v) fetal bovine serum (FBS) in RPMI 1640 medium supplemented with 100 U penicillin G/ml, and 100 mg streptomycin/ml. Cell cultures were maintained in growth medium in which the FBS content was lowered from 10% to 2%. Cell cultures were incubated at 37 C in a humidified atmosphere containing 5% CO2 and 95% air.

For quantitative studies, merozoites were harvested from infected cell cultures by removing the medium and replacing it with Hanks' balanced salt solution without calcium and magnesium. The host cells were then removed from the plastic growth surface by use of a cell scraper. This cell mixture was passed through a 27-gauge needle attached to a 10-ml syringe to rupture host cells. The suspension was then filtered through a sterile 3 J. m filter to remove cellular debris. The number of merozoites in the filtrate was determined using a hemacytometer. The final volume of suspension was adjusted so 2.5×105 merozoites were present for inoculation.

For general maintenance of merozoites, monolayers were examined with an inverted microscope for the development of lesions (areas devoid of host cells caused by parasite replication) in the monolayer or the presence of many extracellular merozoites. Once lesions were observed or many extracellular parasites were present, the monolayer was scraped with the tip of a 5 ml pipette and 1 to 3 drops of the merozoite containing fluid was transferred to 2 flasks of BT cells. Merozoites of S. neurona were passaged in this manner every 3 to 7 days. Decoquinate (lot 7916Z4) was dissolved in DMSO to make a stock solution of 1 mg/ml. Dilutions were made from this stock solution and used in the following studies.

Experiment 1. Merozoites (200,000/flask) of the SN6 strain were inoculated on to cell cultures and allowed to penetrate host cells for 2 hours. The host cells were then treated with 0. 1 microgram/ml decoquinate for 5 minutes or 15 minutes. Control flasks contained merozoites but no decoquinate. The decoquinate containing medium was washed off the infected host cells at 5 or 15 minutes and they were rinsed with Hanks balanced salt solution 5 times to remove any residual decoquinate. Cell cultures were maintained for 6 weeks. No parasites or parasite induced lesions were seen in the decoquinate treated infected flasks at 6 weeks. The control flask was destroyed by this time due to parasite multiplication. These results demonstrated that decoquinate can effectively kill Sarcocystis neurona after a 5 or 15 minute exposure period.

Experiment 2. Merozoites (1 million/flask) of the SN2 and SN3 strains were inoculated separately on to cell cultures and allowed to penetrate host cells for 2 hours. The host cells were then treated with 0.1 microgram/ml decoquinate for 10 or 20 minutes. Control flasks contained merozoites but no decoquinate. The decoquinate containing medium was washed off the infected host cells at 10 or 20 minutes and they were rinsed with Hanks balanced salt solution 5 times to remove any residual decoquinate. Cell cultures were maintained for 16 days. No parasites or parasite induced lesions were seen in the decoquinate treated SN3 infected flasks. Flasks containing the SN2 strain had 5% cytopathic effect (CPE). The control flasks had 25% (SN3 strain controls) to 40% (SN2 strain controls) CPE at this time. These results demonstrated that decoquinate can effectively inhibit several strains of Sarcocystis neurona.

Experiment 3. Merozoites of the SN3 strain were inoculated on to cell cultures and allowed to penetrate host cells for 2 hours. The host cells were then treated with 0.01 (2 flasks), 0.001 (2 flasks), or 0.0001 (2 flasks) microgram/ml decoquinate continuously for 10 days.

Control flasks contained merozoites but no decoquinate. The numbers of merozoites produced were determined at 10 days and a percent reduction in merozoite production determined. Treatment with 0.01 microgram/ml caused a 98% reduction in merozoite production.

Treatment with 0.001 microgram/ml caused a 87% reduction in merozoite production. Treatment with 0.0001 microgram/ml caused a 40% reduction in merozoite production. These findings indicate that there is a dose response to decoquinate and suggests setting a target dose of 0.01 microgram or greater.

The results of Experiments 1,2 and 3 above establish killing activity of decoquinate. Moreover, decoquinate is superior to diclazuril and other conventional agents used to treat EPM because decoquinate kills the EPM-causing parasite more rapidly and at lower concentrations. Novel and unknown to Dr. Lindsay , there are three phenotypes of S. neurona SAG 1, 5, and 6. The phenotypes display different virulence in horses that affect outcome of infctions.

The above data are particularly important considered in view of EPM being a neurologic syndrome in horses caused primarily by infection with Sarcocystis neurona and rarely with Neospora hughesi, and further considering that EPM is the most important protozoal disease of horses in the United States and is present in the Americas wherever the definitive host the opossum is found. The present inventor has considered that treatment of EPM conventionally has often been with pyrimethamine combined with trimethoprim and sulfonamides, which are agents that are known toxic to the horse at required treatment levels.

Thus, prevention is a rational alternative to treatment of clinically ill animals and new effective agents are needed to treat or better prevent EPM. The present inventor has identified such new anti-EPM agents, such as decoquinate and decoquinate levamisole.

Decoquinate is a quinolone antiparasitic agent. Decoquinate inhibits the parasites' mitochondria. Levamisole is an immune modulating agent that may have profound synergistic effects to alleviate signs of EPM in the horse. The experimental data set forth below indicate that decoquinate can quickly kill stages of Sarcocystis neurona in horses and rapidly alleviate clinical signs of EPM. Decoquinate also exerts its anti-Sarcocystis neurona activity at low doses in cell cultures, and is safe in the horse and not readily toxic to other vertebrate species. This indicates it will be safe when used by lay people (i. e. horse owners).

Without limiting the invention to such an example, an example of preventing EPM in a horse not exhibiting any current EPM symptoms would be to add decoquinate in powdered form to the dry feed daily, a daily preventative for EPM in horses. It can be fed alone or in combination with other agents (antibiotics, vitamin supplements, herbal supplements, mineral supplements, etc.) which are fed daily.

Another delivery mechanism would be for feed suppliers to mix decoquinate in the ration. Weekly or monthly administered sustained release formulations of decoquinate also may be used for the prevention of EPM. Horses on preventative decoquinate treatment are expected to be protected against EPM caused by Neospora hughesi, as delivery systems that are preventative against Sarcocystis neurona will be preventative against Neospora hughesi.

Again without limiting the invention to such an example, an example of treating EPM would be as follows. Upon a horse exhibiting an EPM symptom, decoquinate/levamisole is administered to the horse.

To test the idea that decoquinate combined with levamisole would effectively treat and prevent clinical signs of EPM the inventors initiated the following study:

A trial was initiated in which veterinarians conducted neurological examinations and determined a presumptive diagnosis of EPM. In the horses a blood sample revealed antibodies consistent with S. neurona infections or EPM. The phenotype of S. neurona eliciting antibody production was determined for each animal. The animals were treated with decoquinate/levamisole at 0.5 mG/kG and 1.0 mg/kG respectively for 10 days in a compounded paste. A neurological exam was conducted at the end of the treatment period and two to four weeks following treatment. Two to four weeks after treatment antibody levels were again assessed for serum antibody levels to S. neurona by phenotype. Trial outcome was defined as Successful treatment—alleviation of clinical signs of EPM and No change. One hundred and sixteen animals were enrolled in the study of which 52 have completed the trial to date. Successful treatment was found in 49 (94.2%) of the animals. No change was noted in 3 animals (5.8%). For the animals with no change, radiographs revealed an alternate cause of ataxia, cervical vertebral malformation (2) or another musculoskeletal lameness (1). Veterinarians involved in the study elected to start decoquinate therapy at 0.05 mG/kG if the animals had received anti-protozoal therapy in the past and the case was considered a relapse of EPM. For this group of historically relapsing horses, 17 had received Marquis and 2 had received pyrimethamine/sulfadiazine with no change we documented 100% success. In these horses the relapses were attributed to strains of S. neurona that were resistant to anti-protozoal drugs Marquis and pyrimethamine/sulfadiazine which was determined by phenotype. No resistance was identified when therapy consisted of decoquinate/levamisole based on clinical response and antibody titer decline. A group of mildly afflicted horses were placed on prevention dose (0.05 mG/kG) decoquinate alone and noted improvement occurred, although it took longer than the 5 days of therapy with decoquinate/levamisole at 0.5 mG/kG and 1.0 mg/kG. This group (7) continued therapy for 90 days with success obtained in all the horses.

Common structural features and other common properties between decoquinate and the other compounds suggest that the other compounds of similar structure are highly likely to have such killing activity. Thus, these compounds are concluded to have anti-EPM activity, as a prophylactic for preventing EPM and for treating EPM (including treatment of acute EPM), based on the activity of such compounds on EPM-causing parasites. Levamisole is an immune modulator in the horse. While the anti-parasitic effects of levamisole are believed to stimulate the parasympathetic and sympathetic ganglia in susceptible helminthes, it is unanticipated that this drug would affect apicomplexan protozoa directly by this mechanism. At higher levels, levamisole interferes with nematode carbohydrate metabolism by blocking fumarate reduction and succinate oxidation. Levamisole's effects are considered to be nicotine-like in action. The action on apicomplexan parasites is unknown. However the immune stimulating effects on the horse are anticipated to be the mode of action in acute EPM. Sarcocystis neurona was shown by the inventors and others that the effect of infection is a suppression of key cellular events that favor parasite growth in the host. Levamisole's mechanism of action for its immunostimulating effects are not well understood. It is believed it restores cell-mediated immune function in peripheral T-lymphocytes and stimulates phagocytosis by monocytes. Its immune stimulating effects appear to be more pronounced in animals that are immune-compromised.

While horses in the United States, Canada and South America may particularly benefit from the present invention, the invention may be used with regard to horses anywhere.

While the invention above has been discussed with particular reference to horses, it will be appreciated that the invention is not particularly limited and may be used for treating other animals, such as, by way of non-limiting examples, Australian marsupials, arboreal monkey species (including endangered monkey species), sea otters, sea lions, skunks, raccoons, mink, and other animals in aquaria, zoos or farms. Administration of the compounds of FIG. 1 to such animals may aid in preventing fatal toxoplasmosis in highly susceptible animals.

While the invention has been described in terms of its preferred embodiments, those skilled in the art will recognize that the invention can be practiced with modification within the spirit and scope of the appended claims.

We claim:

1. A method of killing apicomplexan parasites. that infect horses, Sarcocystis sp comprising contacting a population comprising Sarcocystis fayeri, Sarcocystis falcatula, Sarcocystis neurona and/or Neospora hughesi with an agent selected from the group consisting of decoquinate, a 4hydroxyquinolone and a naphthoquinone, and/or levamisole a imidazothiazole and salts, esters or derivatives thereof. More specifically apicomplexan parasites that cause equine protozoal myeloencephalitis, Sarcocystis neurona and Neospora hughesi prevention of equine piroplasmas due to babesiosis, Babesia equi and Babesia caballi, and prevention of intestinal coccidiosis caused by Eimeria leuckarti in foals and other equids. In addition, prevention of abortions due to Neospora caninum, Neospora hughesi or Toxoplasma gondii cattle.

2. The killing method of claim 1, wherein said population is in an equid host

3. The killing method of claim 2, wherein contacting includes the step of orally providing said equine host with said agent.

4. A method of preventing or treating equine infections, equine disease, or equine neurological disease or dysfunction, comprising administration to an equine of a pharmaceutically effective dose of an agent selected from the group consisting of decoquinate, a hydroxyquinolone and a naphthoquinone, and/or levamisole a imidazothiazole and salts, esters, or derivatives thereof.

5. A method of preventing or treating bovine abortion comprising administration to an equine or bovine of a pharmaceutically effective dose of an agent selected from the group consisting of decoquinate, a hydroxyquinolone and a naphthoquinone, and/or levamisole a imidazothiazole and salts, esters, or derivatives thereof.

6. The method of claim 4, wherein said agent is decoquinate.

7. The method of claim 4, wherein said agent is a naphthoquinone.

8. The method of claim 4, wherein said agent is a 4 hydroxyquinolone.

9. The method of claim 4, wherein said agent is a imidazothiazole.

10. The method of claim 4, wherein said agent is levamisole.

11. The method of claim 4, wherein said agent is a combination of the above chemicals.

12. The method of claim 4, wherein the disease or neurological disease or dysfunction is associated with infection with an apicomplexan parasite.

13. The method of claim 4, wherein the neurological disease or dysfunction is protozoal myeloencephalitis.

14. The method of claim 4, wherein the apicomplexan parasite is Sarcocystis fayeri, Sarcocystis falcatula, and/or Sarcocystis neurona

15. The method of claim 4, wherein the apicomplexan parasite is Neospora hughesi

16. The method of claim 4, wherein the parasite is Babesia equi and Babesia caballi

17. The method of claim 4, wherein the parasite is Neospora caninum, Neospora hughesi or Toxoplasma gondii

17. The method of claim 4, wherein the parasite is Toxoplasma gondii

18. The method of claim 4, wherein the equine has not yet demonstrated symptoms associated with protozoal myeloencephalitis.
